(19) United States
(12) Patent Application Publication
(10) Pub. No.: US 2015/0178463 A1
(43) Pub. Date: Jun. 25, 2015

(54) SYSTEM AND METHOD FOR THE PROVISION, COORDINATION, AND DELIVERY OF COMPREHENSIVE COPD CARE

(71) Applicant: Temple University, Philadelphia, PA (US)

(72) Inventor: Gerard Joseph Criner, Bryn Mawr, PA (US)

(21) Appl. No.: 14/408,122

(22) PCT Filed: Jun. 14, 2013
(86) PCT No.: PCT/US2013/046005

§ 371 Date: Dec. 15, 2014

Related U.S. Application Data
(60) Provisional application No. 61/659,610, filed on Jun. 14, 2012.
(60) Provisional application No. 61/754,820, filed on Jan. 21, 2013.
(60) Provisional application No. 61/761,970, filed on Feb. 7, 2013.
(60) Provisional application No. 61/768,105, filed on Feb. 22, 2013.
(60) Provisional application No. 61/775,048, filed on Mar. 8, 2013.

(30) Foreign Application Priority Data
Dec. 4, 2012 (US) PCT/US2012/067775

(51) Int. Cl. G06F 19/00; G06Q 50/22; A61B 5/00

(52) U.S. Cl. CPC G06F 19/3431; A61B 5/7275; G06Q 50/22; G06F 19/3418

(57) ABSTRACT

Provided are a method and system for assessing a condition of a patient with chronic condition. The system includes a non-transitory, computer-readable storage device that stores a plurality of variables relating to symptoms being experienced by the patient during an exacerbation of the chronic condition. A baseline component is operable to establish a baseline indicative of a normal condition of the patient with the chronic condition. And a risk assessment component is operable to compare the condition of the patient as determined based on the variables to the baseline to determine a severity of the exacerbation of the chronic condition relative to the baseline.

### CROSS-REFERENCE TO RELATED APPLICATIONS

This application claims the benefit of U.S. Provisional Application No. 61/659,610, filed Jun. 14, 2012; International PCT Patent Application No. PCT/US12/67775, filed Dec. 4, 2012; U.S. Provisional Application No. 61/754,820, filed Jan. 21, 2013; U.S. Provisional Application No. 61/768,105, filed Feb. 22, 2013; U.S. Provisional Application No. 61/775,048, filed Mar. 8, 2013; and U.S. Provisional Application No. 61/761,970, filed Feb. 7, 2013, each of which is incorporated in its entirety herein by reference.

### BACKGROUND OF INVENTION

1. Field of the Invention

The present invention relates in general to the management of a chronic disease or other medical condition, and in particular, to a system and method for the provision, coordination, and delivery of comprehensive care for a chronic obstructive pulmonary disease patient in a telemedicine setting utilizing a patient Anchor/Baseline.

2. Description of Related Art

COPD is a common debilitating disease associated with considerable morbidity and mortality. It affects over 12 million and is the 3^{rd} leading cause of death with a rate of one person dying of COPD every 4 minutes in the United States. Furthermore, COPD is one of the 7-most-common causes of readmission to the hospital in the U.S. and ranks as the 4^{th} leading cause of readmission based on Medicare data. The national readmission rate for COPD is 22.6% and accounts for 4% of all 30-day readmissions.

The reasons for these high readmission rates are because COPD patients are chronically ill, with significant multiple life-threatening comorbidities. In addition, many COPD patients live alone with a high percentage having cognitive dysfunction and depression. COPD patients require complex treatments that include multiple inhaled and oral medications, oxygen therapy and additional interventions needed to treat escalating symptoms that herald an exacerbation.

### BRIEF SUMMARY OF THE INVENTION

The factors and considerations noted above necessitate a comprehensive care management plan that includes intensive education about the nature of the primary disease and comorbidities, ongoing regular contact with a knowledgeable supervising care team and close monitoring of subject adherence to the action plans that triggers prompt recognition of escalating symptoms and timely treatment.

Thus, for close monitoring of subject and adherence to the action plans appointed, all parties coordinating care must remain knowledgeable and up to date of the current patient status. In a telemedicine system where a patient may receive treatment in distant facilities or in the confines of their own home, there needs to be an established workflow system so that all parties remain informed and knowledgeable in the decision making process.

Accordingly, there is a need in the art for a method and system that can be utilized by patients and a clinical team in a telemedicine system setting so that the best possible care is provided. In a large hospital system setting, it is often unnecessary, arduous, and impractical to monitor each and every chronologically ill patient. Thus, the present telemedicine system provides care to multiple hospital systems with diverging patient populations and is adaptable to different hospital systems because it is reflexive to change, and encompasses providing immediate care and patient information.

When a patient's symptoms are worsening indicating an acute exacerbation, it is possible to improve a patient's status via clinical intervention through a variety of medication or treatment pathways. The use of different medications or treatment pathways is prescribed only if the patient and certain symptoms are recognized in the individual. For example, the presence of purulent sputum can be an indicator of an exacerbation in COPD patients by pulmonologists, but the presence of purulent sputum may read as a different indicator by a Primary Care Physician (PCP) who does not have specialty training. This aforementioned situation could lead to possible redundancy in treatment or the misdiagnosis of a possible debilitating episode.

Thus, the present system and method address the presence of an exacerbation in a timely and efficient collaboration between clinician, physician, therapist, patient, and other parties involved in the health-related decision making process.

The present system and method provide a new and useful system for clinicians in a telemedicine system setting to provide efficient and effective care to patients, in which the invention serves as a peripheral device to (or integrates with) an automated reference Baseline/Anchor Exacerbation Score.

The present system and method, in effect, offer an individualized, customizable treatment pathway and workflow system in a telemedicine setting to provide inclusive and extensive treatment on per patient to per patient cohort to per hospital system scale. Furthermore, present system and method permit the clinician to customize the COPD telemedicine system to allow the clinician or clinical team to customize the flow of information to their choosing in such a way that certain information is automatically fed to or bypassed to certain parties. The method and system can allow the flow of information to be sequential or instantaneous, or both. And, the flow of information can proceed in a hierarchical or circuitous route.

To maintain a method of efficacy, it is necessary for the clinical team to monitor the patient's daily symptoms, medications, and allergens from the point of registration of patient into said system, to divergence from Baseline/Anchor, to automated recommendation of medications, to the return of patient to their reference Baseline/Anchor. The invention in effect converts the compiled Baseline/Anchor information and automatically submits the patient information based upon divergence from baseline and presence of symptoms to the most pertinent clinical team members and the respective third parties (caregivers, relatives, PCPs, etc.).

Accordingly, there is a need in the art for a method and system that can be utilized by a clinician or clinical team caring for a patient with a chronic illness or illnesses, to establish an automated system that can assess symptoms associated with a chronic illness following an exacerbation intervention, and will automatically feed the patient data to the necessary parties associated, based upon the presence of certain symptoms and their associated severity, optionally as those symptoms compare to a baseline established for the patient.

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1. Shows an embodiment of a computer system for assessing a condition of patient with a chronic illness

FIG. 2 shows an embodiment of a check-in interface displayed to a user of a hand-held device;

FIG. 3 shows an embodiment of a symptom interface providing a menu of symptom samples selectable by a patient to transmit any symptoms to a server over a communicated network;

FIGS. 4A-F show illustrative embodiments of computer-generated displays generated to facilitate review and performance of a treatment path;

FIG. 5 shows an illustrative embodiment of a linear information flow pathway, which requires each step to be completed before the next step can be performed;

FIG. 6 shows an illustrative embodiment of a circuitous information flow pathway, which involves different steps being performed concurrently;

FIG. 7 shows an illustrative embodiment of a computer-generated notification list presenting different categories/types of notifications, and who receives each such notification before, during, and after an intervention;

FIG. 8 shows an illustrative embodiment of a computer-generated notification creation display that can be used to create or edit a notification and a recipient of a notification;

FIG. 9 shows an illustrative embodiment of a computer-generated user interface to be used for creating one or more intervention steps or actions to be taken by an individual or individuals in sequence or concurrently;

FIG. 10 shows an illustrative embodiment of a computer-generated user interface for the creation or editing of an intervention, a party assigned to complete an intervention step or action, and a requirement for a signature;

FIG. 11 shows an illustrative example of a computer-generated presentation of recommended treatments (e.g., go to ER, go to outpatient clinic) based on a deviation from a baseline and/or underlying chronic condition and/or severity of exacerbation, and participants coordinating care around a patient to complete an intervention;

FIG. 12 shows an illustrative embodiment of a computer-generated user interface for editing a type of treatment (e.g., go to ER, go to outpatient clinic), intervention steps, and the information that is ultimately sent to the patient;

FIG. 13 shows an illustrative embodiment of a computer-generated user interface for configuring treatment paths based on disease type (e.g., COPD vs congestive heart failure), stage or severity of a patient's underlying condition (e.g., GOLD 1 Vs Gold IV COPD) and the duration over which a patient is reporting elevated symptoms (e.g., over a period of 1 or more days) based on deviation from a baseline and/or exacerbation score;

FIG. 14 shows an illustrative embodiment of a computer-generated user interface for editing treatment paths and escalations of treatment over time at a system level; and

FIGS. 15-18 show an illustrative embodiment of a computer-generated display for editing treatment paths and escalations of treatment over time at individual patient level.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

An illustrative embodiment of the present method and system is described herein for: determining the correct method in onboarding of patients into said system, setting thresholds of predicative measures in evaluating acute exacerbations, setting reference baselines/anchors used to assess the status of a chronically ill patient, determining optimal treatment pathways and information flow in providing comprehensive care for use in assessing and treating the condition of patients with a chronic pulmonary illness, specifically chronic obstructive pulmonary disease (“COPD”). The baseline is also indicative of a likelihood that another exacerbation will be experienced by the patient in the future, or can optionally reflect an expected frequency at which the patient can expect to experience future exacerbations. It is to be understood, however that the present method and apparatus are not so limited, and can be utilized to assess the condition of a patient suffering from any chronic illness in an ongoing basis. Once the comprehensive treatment pathway has been established, the treatment pathway can be reassessed after a period of time has passed since the treatment pathway was initially established to reflect efficiency in a dynamic setting.

The reference baseline of the individual patient afflicted with COPD can be used in a clinical setting. A patient or clinician can manually enter, or a medical device sensing a quantity or quality of a parameter relating to the patient can transmit values of variables into an algorithm that can be manipulated by a computer processor, which can optionally be remotely located from the patient over a communication network. The computer processor can transmit a set of pre-designated questions to be answered by the patient, and the answers used to input variables to the algorithm for establishing and updating the reference baseline. This baseline, however, can optionally be required to be established as a result of an in-person examination of the patient by a medical professional (e.g., physician, clinician, etc. . . . ). In other words, a medical examination of the patient by the medical professional can be a prerequisite to the establishment of one or more initial score values representing thresholds that, when surpassed during a subsequent exacerbation experienced by the patient, can result in the recommendation of a predetermined course of treatment by the computer system 10 as described herein.

The comprehensive treatment pathway of the individual patient afflicted with COPD can be used in a clinical setting. Furthermore, the treatment pathway system serves as a peripheral device to (or integrates with) an automated reference Baseline/Anchor system. The reference baseline system, which is an automated scoring system to provide an updated patient status, allows the patient or clinician to manually enter, or a medical device sensing a quantity or quality of a parameter relating to the patient can transmit values of variables into an algorithm that can be manipulated by a computer processor, which can optionally be remotely located from the patient over a communication network. The computer processor can transmit a set of pre-designed questions to be answered by the patient, and the answers used to input variables to the algorithm for establishing and updating the reference baseline/anchor.

In the coordinated care system, onboarding of patients into the system may be done so by a clinician or patient via a wireless network. Key categories of information needed in the onboarding process are contact information, third party contact information, medical information, and clinical contact information. For contact information it is necessary to attain patient name, age, gender, birthdate, phone number, email, and status of the patient in the system (active/inactive). Third party contact information includes primary care physician information, caregiver information, and pharmacy information. Medical information includes the allergies, last discharge date, and medical record number. Clinical contact information includes specialist physician(s), respiratory therapist(s), physical therapist(s), case manager(s), primary care physician, and any other assigned clinical personnel email, phone number, or point of contact. Once the essential information is collected, the patient can be onboarded into the system with a unique username and password with HIPAA compliance.

An alternate embodiment of the method and system take into consideration differing illness severities, optionally in addition to other illnesses (co-morbidities) that may be inflicting the patient in addition to COPD in establishing the reference baseline. Each patient can input the value of variables (e.g., manually, or through the use of a medical device) to establish a normalized baseline reference point of patient wellness that takes any co-morbidities into account. In particular, a starting point can be established for each individual patient for use in any such system in which the medical status is being monitored, collected and analyzed. This starting point, which can be determined after a predestined period of time, can serve as a reference baseline indicating overall patient status and wellness from the outset of use of the present method and system.

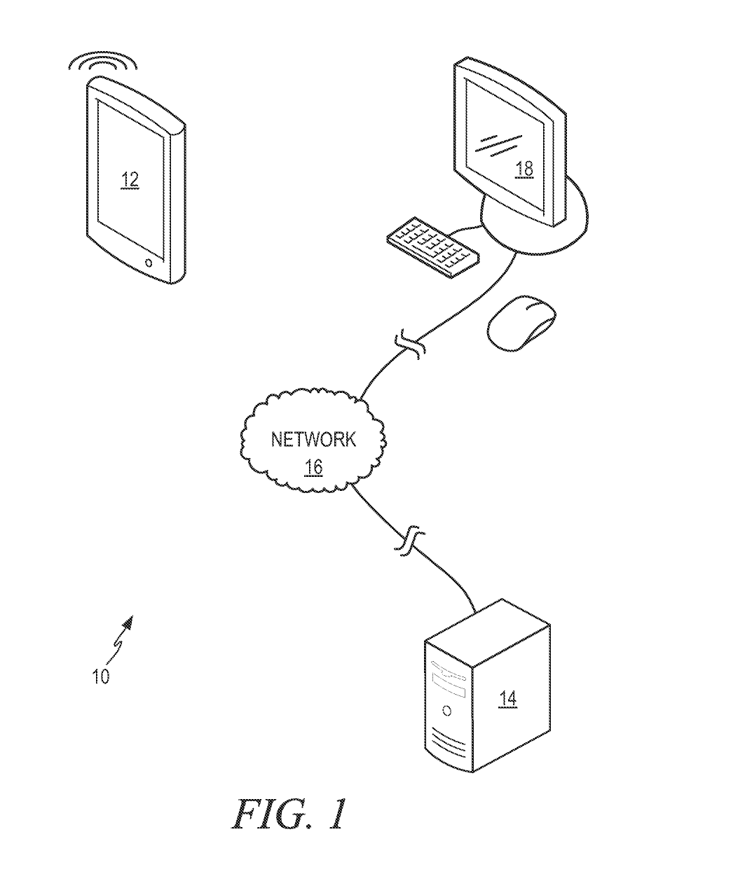

FIG. 1 shows an illustrative embodiment of a computer system 10 that can be utilized to perform the method of assessing the condition of patients with a chronic illness. As shown, the computer system 10 includes a tablet computer 12 operatively connected to communicate with a server 14 over a communication network 16. Although shown and described in the illustrative embodiments as a tablet computer 12, a user computer can be any suitable computing device that can present the user with a form-based interface that can be used to enter responses to questions and other variables to be used in the algorithm such as a desktop computer terminal, laptop or notebook computer terminal, portable handheld device such as a cellular telephone or smartphone, and the like.

The communication network can include components of a local area network (“LAN”), a wide area network (“WAN”) such as the Internet, or a combination thereof. The server 14 is a network-connected terminal with a non-transitory computer-readable memory for storing information input by patients and received over the communication network 16. The server can also optionally be programmed (e.g., with Apache HTTP Server software) to function as a database server, file server, mail server, web server, etc. . . . to serve content over the communication network and facilitate the entry of data by users of the tablet computer 12 to establish, update and otherwise interact with the reference baseline as described herein. Computer-executable instructions executed by at least one of the tablet computer 12, server 14 and a clinician terminal 18 described blow can embody a baseline component.

The computer system 10 also includes a clinician terminal 18 that can be used by authorized parties involved in the provision of healthcare to patients to view patient data, or at least a comparison of patient data to the reference baseline. Like the tablet computer 12, the clinician terminal 18 can be operatively connected to communicate with the server 14 over the communication network 16. According to such a network architecture, the tablet computer 12, the clinician computer, or both can be remotely connected to the server 14 over the network 16, instead of being locally and directly connected. Thus, a patient can use the tablet computer 12 at home or other convenient location with Internet access to input the data concerning an exacerbation to seek help in determining whether to seek in-person medical attention during a visit to a healthcare facility such as a hospital.

The computer system 10, or portion thereof, can establish, identify, quantify, measure, compare, and update a baseline signifying a “norm” or “normal” levels that represent an overview, sketch, or representation of a chronic disease patient's condition, including demographics, symptoms, characteristics, and health generally. The “norm” for a patient with a chronic illness can be considered to be the condition of the patient in the absence of an exacerbation, but including symptoms that are expected of a patient with such an illness that cannot be cured. The embodiments used herein to describe the system and method reference a chronic pulmonary illness, specifically COPD as an example, but the chronic illness can be any long-term, and optionally incurable condition.

The tablet computer 12 can be utilized by a patient to enter patient information specific to that patient's COPD condition to establish an Initial Baseline for that patient. The Initial Baseline for a patient is the starting point for reference and later comparison to subsequent evaluations of the patient's COPD condition. The Initial Baseline is determined by obtaining and combining variables that are intrinsically, directly, indirectly, interactively with other variables, or otherwise related to the COPD condition of the patient or the patient's general health. In an illustrative embodiment, the variables used in the determination of the Initial Baseline include a combination of one or more types of variables. Non-limiting examples of such variables include demographic (e.g. gender, age, weight), behavioral (e.g. smoking use, exercise, diet), therapeutic (e.g. medications, oxygen use, therapies), diagnostic (e.g. primary diagnosis, co-morbidities), hospitalizations, medical history, genetics, objective/observed symptoms (e.g. temperature, blood pressure, FEV), and subjective/reported symptoms (e.g. presence of cough, sputum color and quantity). The variables may be located or obtained from a variety of sources, non-limiting examples of which include, physical/paper records, electronic health or medical records, databases, diaries, journals, computers, devices, entries, office visits, telephone calls, emails, texts, smartphone applications, patients, family members, physicians, nurses, healthcare professionals, medical supply companies, or pharmacies. For embodiments that obtain the variable values from electronic sources (e.g., electronic medical records, databases, emails, etc. . . . ) the server 14 can be configured to automatically recognize and extract pertinent information based on International Statistical Classification of Diseases and Related Health Problems codes (also referred to as ICD-9 codes). The server 14 or other portion of the computer system 10 can execute computer-executable instructions to perform any of the actions described herein, including the recognition of ICD-9 codes and the extraction of the corresponding data. For embodiments where the variable values are obtained from non-electronic sources, the data can be manually entered into the computer system 10. The system and method require, prompt for, request, obtain, utilize, store, or otherwise identify the date and time that each variable was observed, reported, measured, or otherwise obtained.

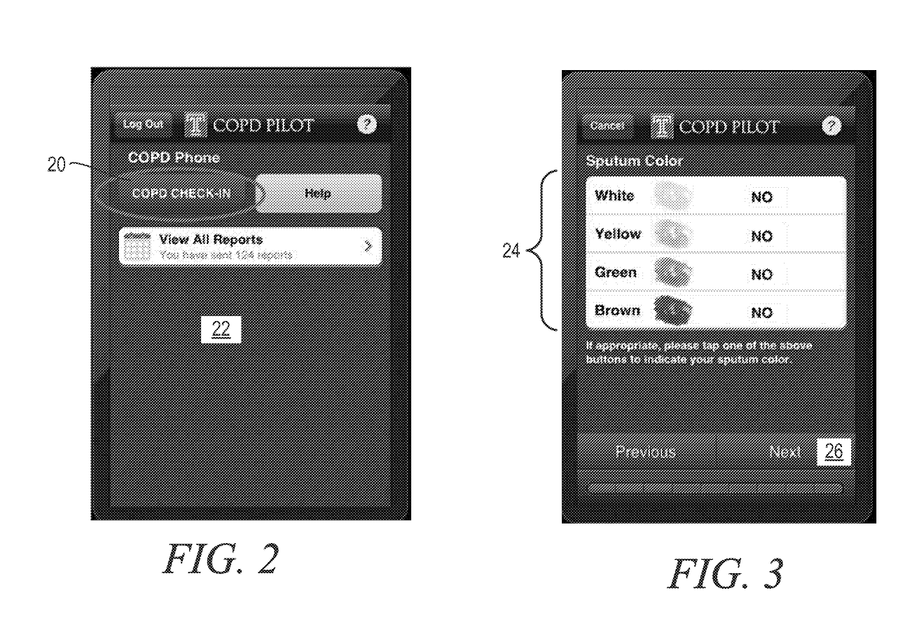

The user interfaces presented by the tablet computer 12 to elicit the patient data from the patient are described with reference to FIGS. 2 and 3. In FIG. 2, the user can select a “check in” option 20 from the check in screen 22 upon experiencing an exacerbation to begin an assessment of the exacerbation and determine whether in-person healthcare is warranted by the exacerbation. Access to the check in option 20 and/or the check in screen 22 can optionally be protected by a password or other security feature to protect any potentially-confidential patient information that may reside on, or otherwise be accessible via the tablet computer 12.

During the check in procedure, the information for determining the Initial Baseline can be input by the user into the interfaces presented by the tablet computer 12. An algorithm can be provided to the tablet computer 12, server 14, clinician terminal 18, or distributed amongst more than one computer, to assign varying degrees of weight or preference to some variables more than others. In a non-limiting example, one or more variables may be combined by way of one or more subcalculations before the final overall calculation is performed. In another non-limiting example, certain components of the calculation may be dependent upon results obtained by one or more subcalculations or one or more variables. The weights assigned to each variable can vary, and be edited by an authorized user. In one non-limiting example, the system and method utilize the most recently obtained data for each variable as identified by any associated date/time stamps in determining the Initial Baseline, updating the value of the Initial Baseline, or obtaining information to compare to the Initial Baseline. In another non-limiting example, the system and method provide an alert if one or more variables or data do not meet a particular threshold or criteria (i.e. if the data for one or more variables are considered “out of date” as defined in the system), prompting the user to update their patient data so the Initial Baseline can remain current. In one example, the threshold, criteria, or other limit is a variable defined and editable by an end user. The calculation, variables, and other components used to identify the Initial Baseline may be altered, updated, amended, or otherwise edited periodically, manually, or automatically.

An example of the type of patient information collected during a check in procedure can include information concerning sputum coughed up by the patient during an exacerbation. To assist the patient in entering a proper description, the sputum screen in FIG. 3 can present a menu 24 of different colors commonly encountered by patients for comparison purposes. The patient can enter the appropriate selection by touching the display of the tablet computer 12 and selecting a “Next” soft key 26 to proceed to the next question.

The illustrative example appearing in FIG. 3 is but one example of a symptom that can be qualitatively/quantitatively entered into the present computer system 10. Other embodiments may utilize pre-defined questions to elicit any desired information pertinent to the assessment of an exacerbation from the patient. The questions can seek to elicit information pertaining to at least one of the following conditions: breathlessness (e.g., on a scale from 1 to 10), the excretion of sputum or other substance (e.g., color, consistency, volume or other quantity; expiratory flow (e.g., peak flow measurements), fever, coughing, wheezing, sore throat, nasal congestion. According to other embodiments, the patient information to be included in the assessment of an exacerbation includes at least one of: COPD classes A, B, C and D based on number of acute exacerbation COPD (“AECOPD”) episodes in the past year, MRC dyspnea class and GOLD stage; severity of AECOPD episodes in past year (e.g., home treated, ER treated, hospitalized, treated in ICU, etc. . . . ); use of mechanical ventilation—invasive or noninvasive; use of supplemental oxygen at home, criteria for home oxygen use; receipt of vaccine for flu and/or pneumococcal; evidence of pulmonary hypertension, whether the patient is compliant or noncompliant with meds; whether the patient has been hospitalized or visited an emergency room or other urgent care facility within a predetermined number of days (e.g., within last 30, 60 or 180 days); presence or absence of hypercapnia; whether the patient is a current smoker or has a history of smoking; Medical Research Council (“MRC”) breathlessness scale score; the patient's Forced Expiratory Volume that has been exhaled at the end of the first second of forced expiration (“FEV₁”); whether the patient suffers from obstructive sleep apnea (“OSA”); the patient's obesity; Diabetes Mellitus (“DM”); osteoporosis; whether the patient lives alone; the saturation level of oxygen in the hemoglobin (“SaO₂”); gender; body mass index; Body Mass Index/Obstruction/Dyspnoea/Exercise Capacity (“BODE”) index score; answers to the St. George's Respiratory Questionnaire (“SGRQ”); SF-36; whether the patient suffers from coronary artery disease/congestive heart failure (“CAD/CHF”); chronic bronchitis; percent emphysema (e.g., >35%); whether the patient has an airway wall thickness (“AWT”) greater than a threshold thickness (e.g., AWT >1.75 mm); medications (e.g., tiotropium, inhaled corticosteroid, salmeterol, formoterol, combination of long acting beta agonist and inhaled steroid, statins, chronic azithromycin use, chronic systemic steroid use (daily use >2 weeks), etc. . . . ).

After a patient has been successfully onboarded into the system, the clinician/clinical team can determine how they would like to weight the reported symptoms and measurements. In a non-limiting example, the “Criner” algorithm and system allows patients to answer questions pertaining to dyspnea, sputum quantity, sputum consistency, sputum color, body temperature, peak flow measurements, and the presence of a cough, wheeze, sore throat, as well as nasal congestion. In this system, patients are awarded points based upon the presence of symptoms and their movement away from baseline/anchor values. In the comprehensive care system, the clinician or the clinical team may adjust what symptoms are to be reported by the patient and the point awarding scale to determine the severity or presence of an impending exacerbation. Furthermore, the symptoms maybe self-reported by the user, entered by a caregiver or clinical personnel, or automated by device. For example, the clinician or the clinical team can optionally require the patient to enter values corresponding to sputum quantity, color and consistency, as well as body temperature to generate a score ranking the severity of the exacerbation, which is to be compared to the baseline to determine the next steps in the patient's treatment.

After the clinical team determines what symptoms are to be measured and how to weight the reported symptoms, the clinician or clinical team must then decide on how they would like to set the baseline/anchor parameters for the patient cohort they are treating. The baseline/anchor in the comprehensive care system is the “Normal” status of the patient, which is determined after a period of time (i.e., 14 check-in days). The baseline/anchor methodology is highly important to the comprehensive care system model because it creates an individualized patient scoring system unique to the patient and their chronic disease. Therefore each patient is vastly distinct in his or her status of “Normal”. In the “Criner” system, baseline/anchors are computed after a predetermined period (e.g., 14 check-in days) has expired to determine the mean or mode of reported patient symptoms. In the comprehensive care model, the range of check-in days they would like the system to compute the clinical team determines the baseline/anchors for. It is important to note that Anchors may be recalculated after a number of days to establish a more current status of the patient. In a non-limiting example, the anchors calculated in the Criner system are done so every 90 days after the initial anchor is calculated after 14 days.

After setting the appropriate anchors, the clinician and/or clinical team must then determine treatment pathways. Treatment pathways are the pre-determined treatment recommendations for patients. The treatment pathways in the comprehensive care system differ greatly for patients under constant clinical surveillance than for those patients who are not. For patients under direct clinical surveillance, it is up to the clinician to determine the correct method of treatment. Under this direct clinical surveillance, patients receive clinical interventional treatment through the use of a communicative device (i.e., phone, computer, tablet). For the patients not under direct clinical surveillance, the pre-determined treatment pathways are a triage system embedded in the comprehensive care system configured by the clinician/clinical team in charge. The treatment pathways allow patients to be automatically filtered throughout the health system to the appropriate coordinated care clinical team member based upon many non-limiting variables including symptom score, duration of symptom score days, and disease stage. The treatment pathways are configurable in that the clinician/clinical team can decide the method in which the patient may be directed or alerted based upon their reported symptoms. For example, in a system in which the patients are reporting symptoms resembling a severe exacerbation over 3 days, the clinician or clinical team may decide to notify the patient population experiencing these symptoms to call their clinical observer directly. In the same scenario of a patient population reporting symptoms resembling a severe exacerbation over 3 days, the clinician or clinical team may decide to notify the patient population experiencing these symptoms to immediately report to the nearest clinical emergency department through automated message. The treatment procedures may change in correlation to Acute Exacerbation score and GOLD stage. In a proposed system, there may be multiple treatment pathways to accompany the vast and unique patient populations. The treatment pathways maybe uniquely different dependent upon the AE score, duration, and treatment procedure from hospital to hospital due to limiting and non-limiting factors and these treatment pathways can be manipulated on a patient, to patient cohort, to health system basis.

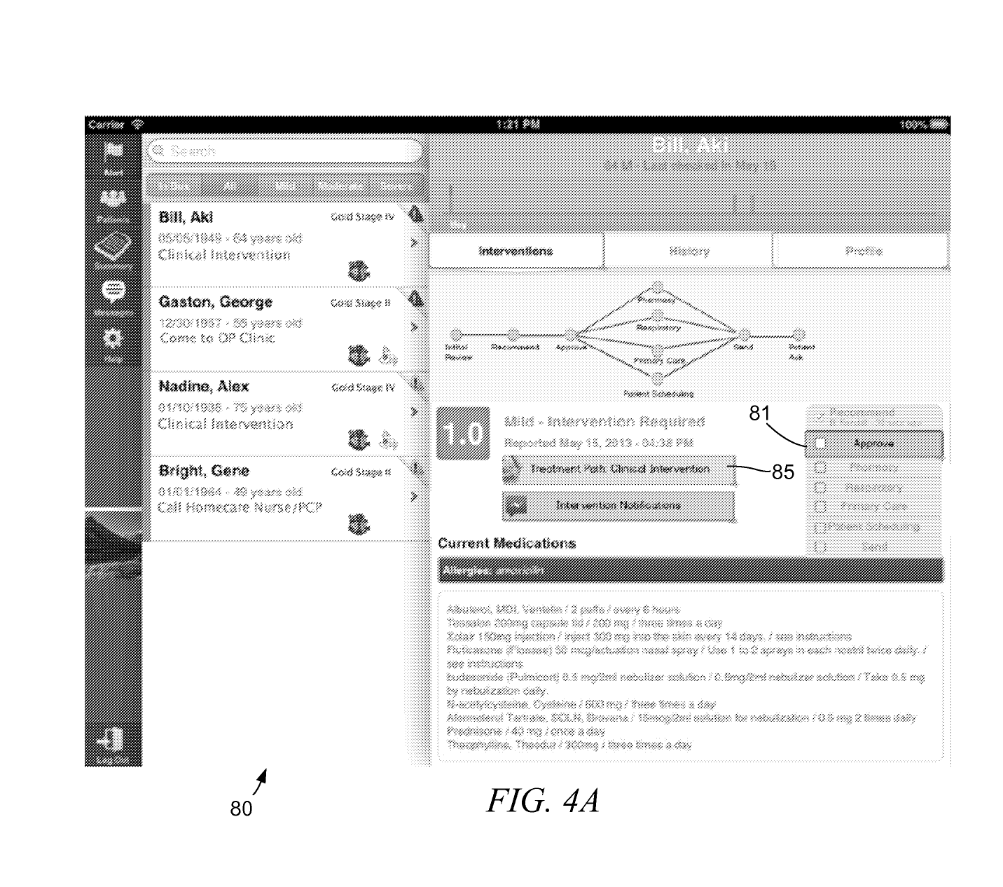

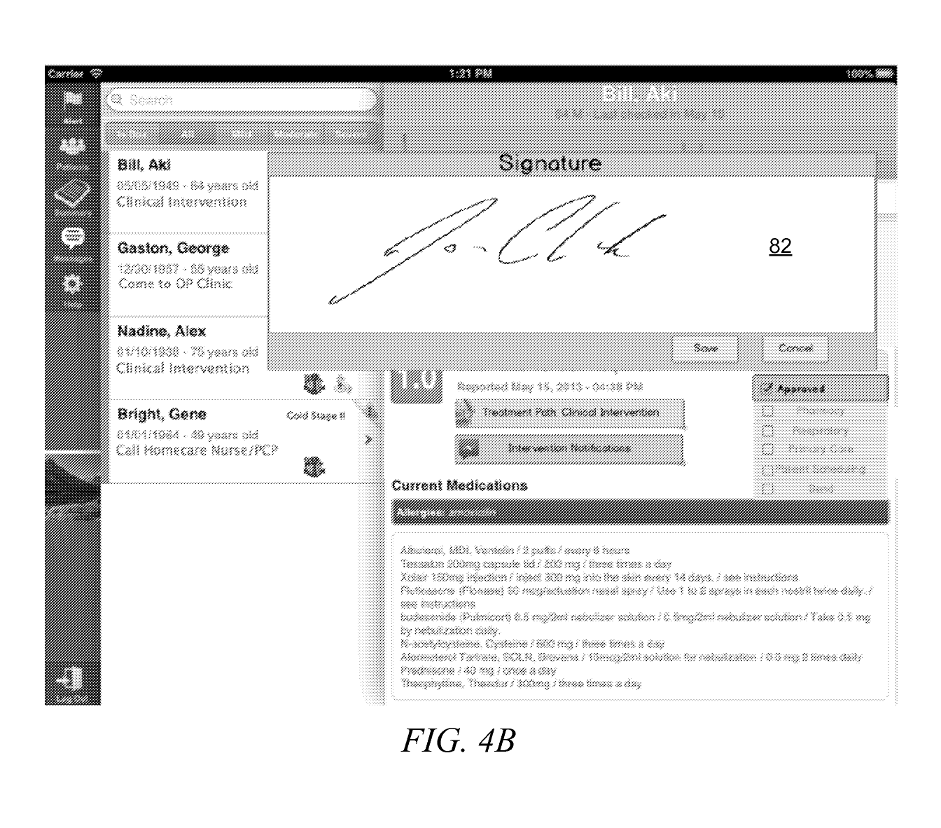

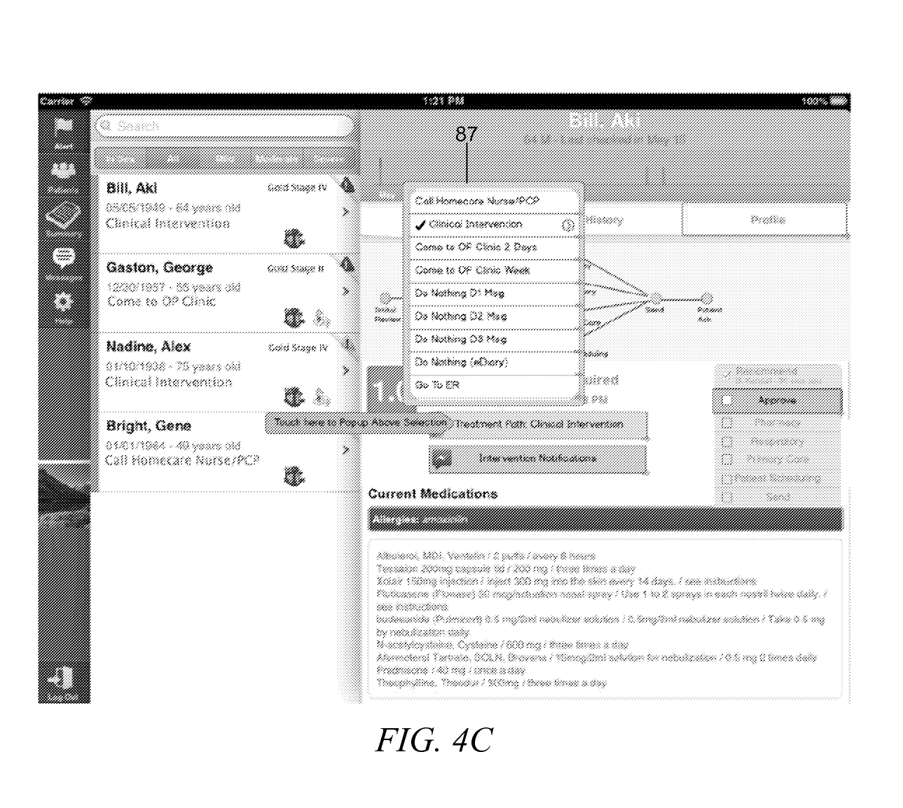

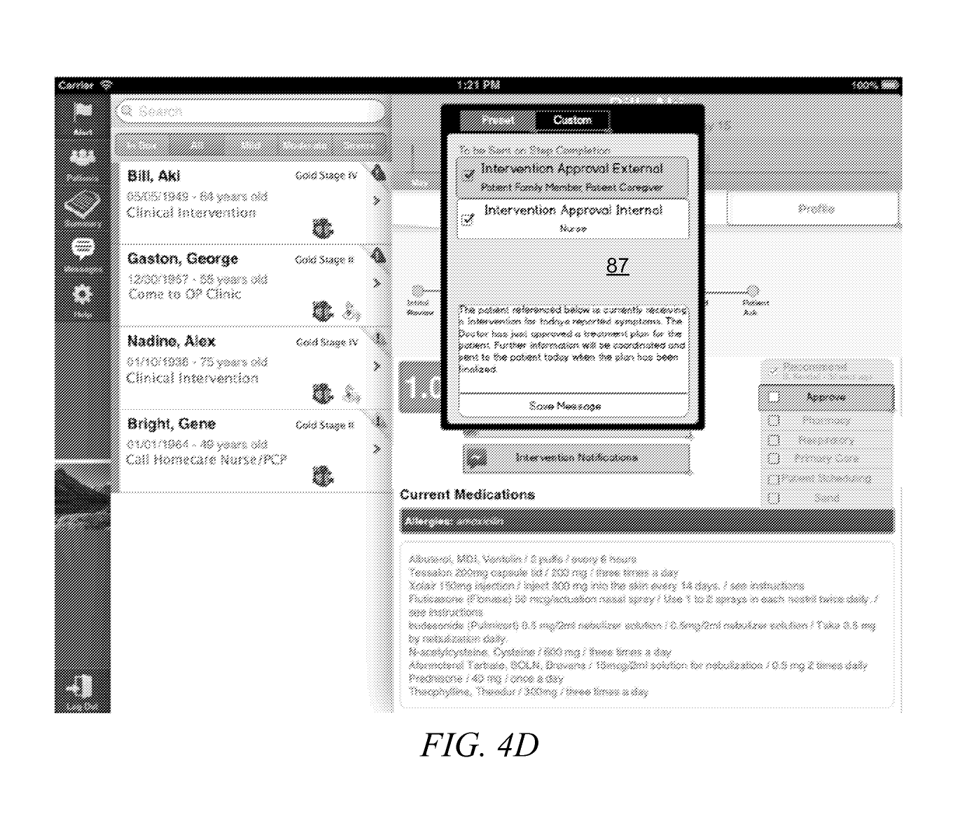

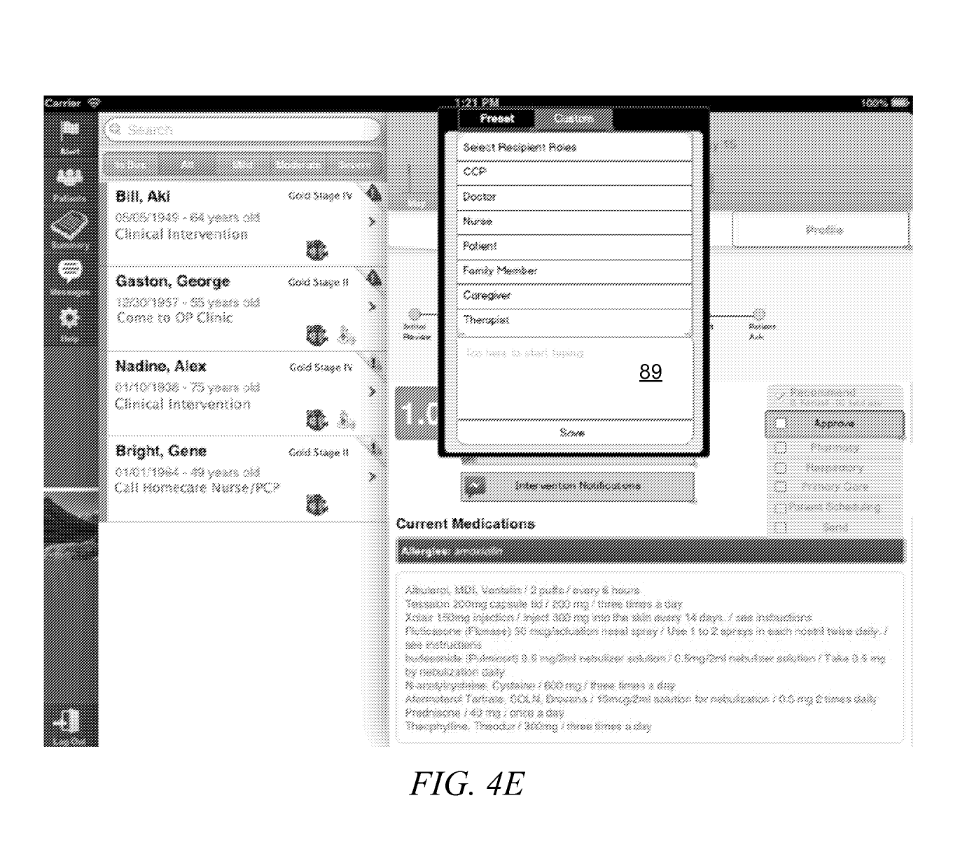

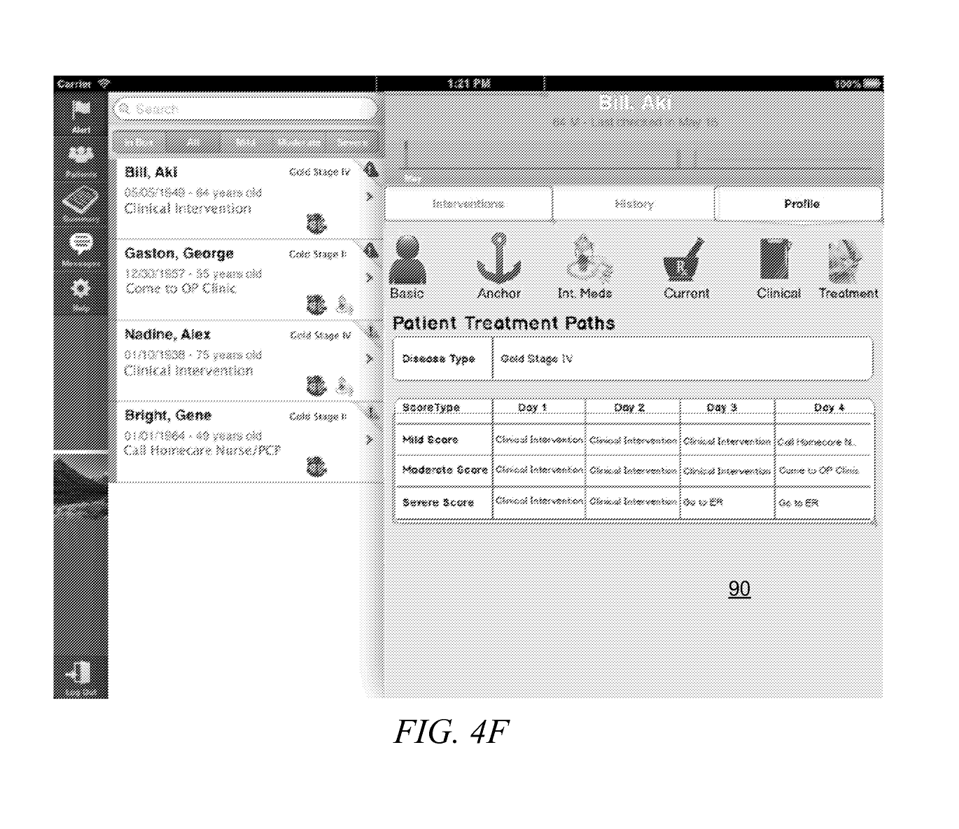

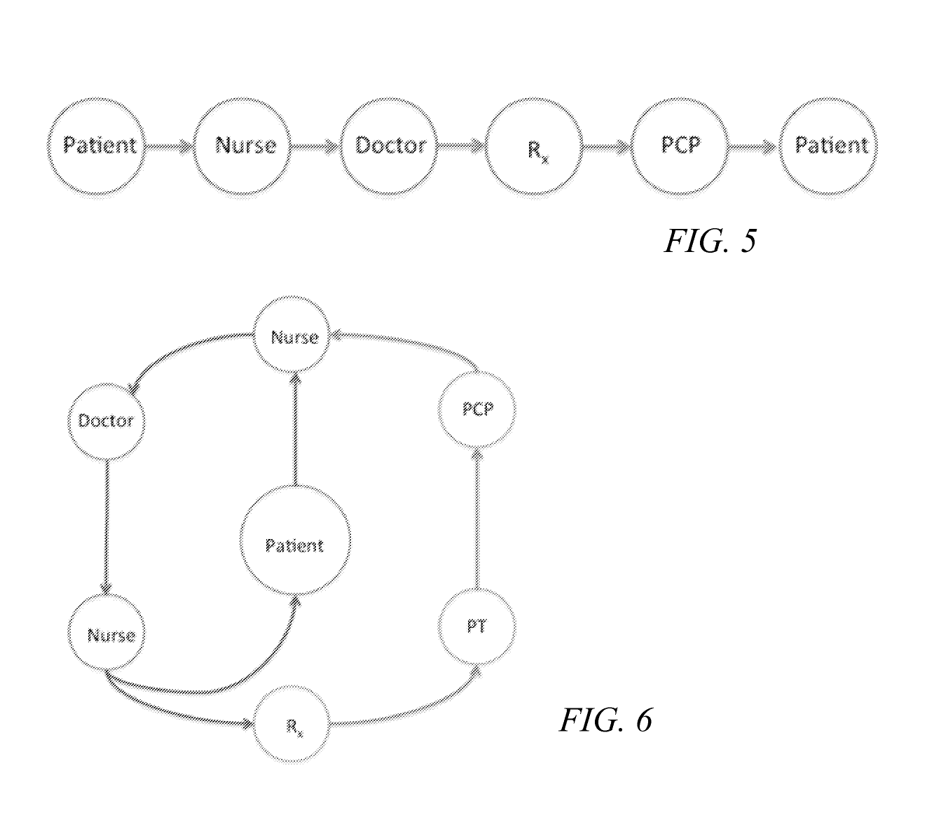

FIGS. 4A-F illustrative various embodiments of treatment options included in a predetermined treatment pathway that can be utilized to perform the method of alerting a patient for treatment after the presence of exacerbation like symptoms after 2 days. As shown in FIG. 4A, which is a screen 80 that can be presented to a nurse, for example, via the clinician terminal 18 (FIG. 1) for example, an exacerbation score of “1.0” calculated by the computer system 10, or portion thereof, was assigned to indicate the severity of the exacerbation based on the patient's input into the tablet computer 12 as described in WIPO International Patent Application No. PCT/US12/67775, which is incorporated in its entirety herein by reference. A configurable step in the comprehensive care system is the determination of information flow. The information flow in the comprehensive care system is the chain of events that arises after a patient has reported their symptoms to the clinician or clinical team. The information flow is configurable and can be maintained in a linear or circuitous, and stagnant or continuous mode. In a linear system, schematically depicted in FIG. 5, information is passed from one acting party to the next after one party has completed their duty; once they receive notification that the preceding party has completed their action the next actor will complete their task and submit their completed action to the next designated person in the system automatically or manually. Once all parties have successfully completed their actions, the information flow is complete.

In a non-limiting example, if a patient is reporting symptoms resembling an exacerbation, the patient reports the symptoms, the nurse is notified of the increase in symptom severity and recommends treatment to the attending physician. Based on the displayed score in FIG. 4A, which also takes the patient's baseline into consideration, “Clinical Intervention” was determined by the nurse. The physician reviews the recommended treatment selected by the nurse and either approves (FIG. 4B) or edits (FIG. 4C) the treatment to send back to the patient, the patient then is notified of the recommended treatment and takes action, completing the required steps in the information flow. The physician, for example, caring for the patient can check the “Approve” box 81, causing a signature field to appear as shown in FIG. 4B. The physician or other authorized party can input a signature or other marking into the signature field 82 indicating the approval of the treatment recommended by the computer system 10. The physician can also select the Treatment Path option 85 to display a menu 87 of selectable options for the treatment of this particular exacerbation to edit the recommendation of the nurse. In response to selection of the Treatment Path option 85 the computer system 10 can limit the displayed options to those that belong in the category of “Clinical Intervention”, or can include all available treatment options.

FIGS. 4D and 4E show an illustrative example of a Preset notification menu 87 allowing the clinician to issue preset intervention notifications (FIG. 4D) and a Custom notification window 89 allowing the clinician to issue a custom intervention notifications (FIG. 4E). As shown, the notification can be designated to be transmitted to at least one, and optionally a plurality of intended recipients who should at least be aware of, and optionally involved in the recommended treatment of a particular exacerbation, including the patient.

FIG. 4F illustrates an illustrative embodiment of a screen 90 that can be accessed by a treating physician or other authorized party to customize the treatment paths for specific patients.

FIG. 5 shows an illustrative embodiment of a linear information flow pathway. As discussed above, information is passed from one acting party to the next after one party has completed their duty; once they receive notification that the preceding party has completed their action the next actor will complete their task and submit their completed action to the next designated person in the system automatically or manually. Once all parties have successfully completed their actions, the information flow is complete.

In a circuitous system, shown in FIG. 6, information is passed from one acting party to multiple parties regardless if they are an acting party or an observing party. The acting parties work in the same fashion as the linear system. Observers in the circuitous system receive information but are not required to act on the notification. Furthermore, some parties in the information flow system can be omnipresent but never require any action. An example of an omnipresent observer is a physical therapist, they may never be required to act on recommending treatment, but may need to be involved in the flow of information to remain knowledgeable of their patient cohort. For example, if a patient reports symptoms that exemplify an exacerbation in circuitous information flow system, the exacerbation alert will be sent to all acting/observing parties. The first person to act will then push their report submission to the next acting party. The information flow will continue in this manner until all parties have completed their tasks. The continuous workflow system is a system in which there is continuous feedback and activation by all acting parties to provide greater coverage of care.

The stagnant workflow system is only activated when the starter activates the workflow either through an automated or manual alert. In this non-limiting example the starter is the Patient, who will send out an alert, which is then acted upon by the next party in line—in this case the nurse—who then acts upon the signal and pushes it to the physician, who proceeds to push it down the line until all parties have completed their actions. Similar to neurons acting in a body, if the hand feels the sensation of heat, it will send a signal to the brain telling the hand to recoil. In this sense, the stagnant workflow system is very similar—the acting parties are only notified when the system recognizes the patient is in alert mode and needs immediate action. Only when the patient triggers an alert, will the stagnant workflow system begin.

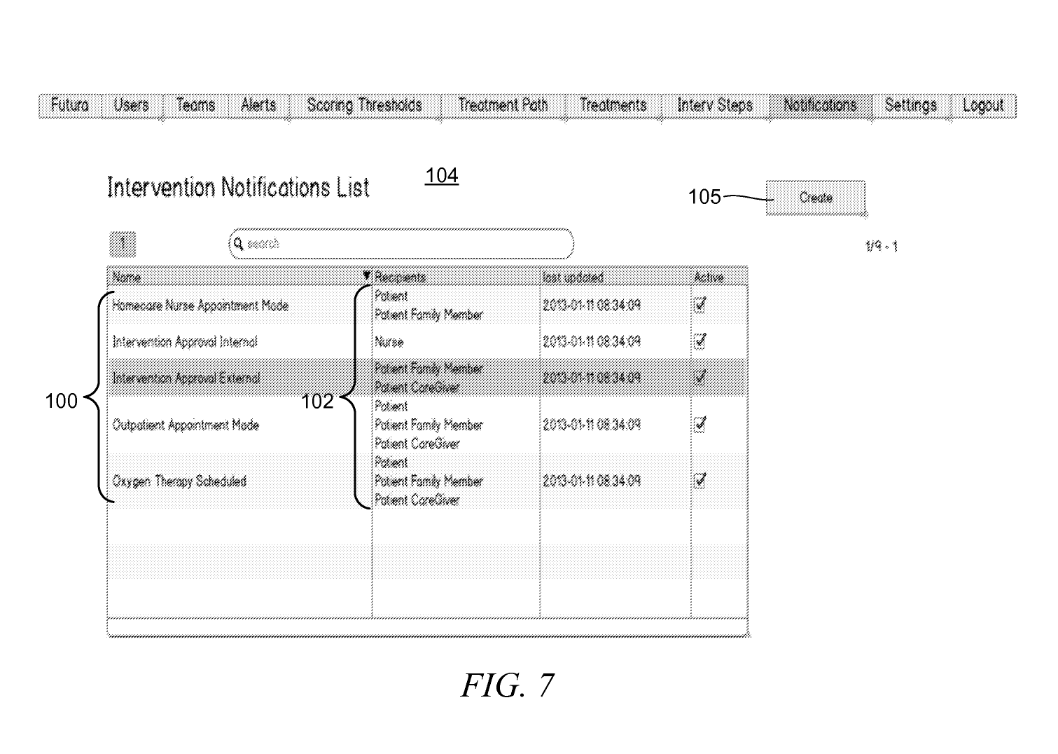

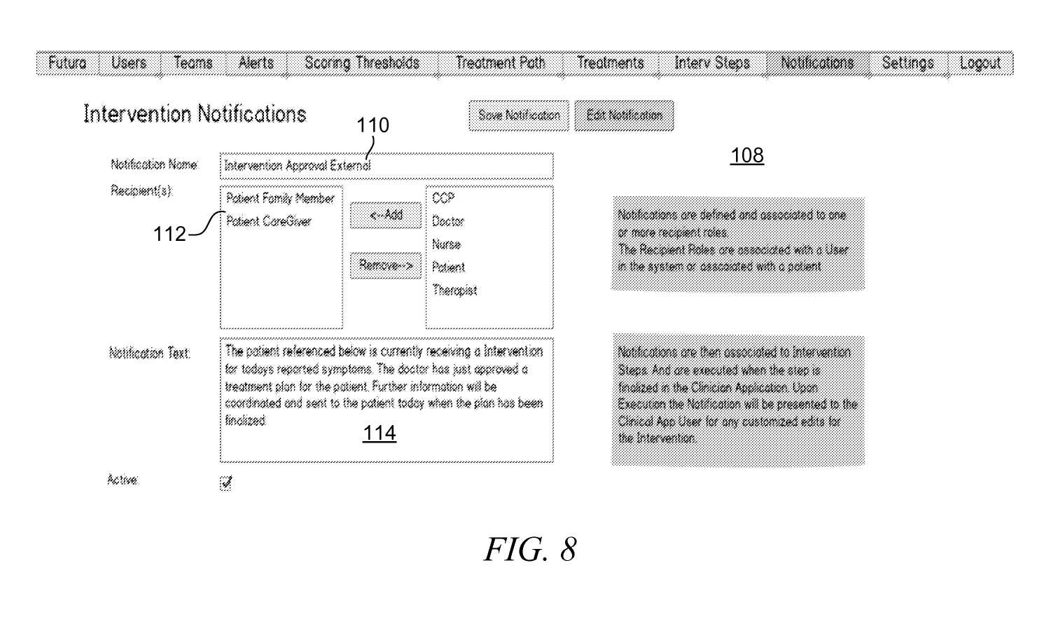

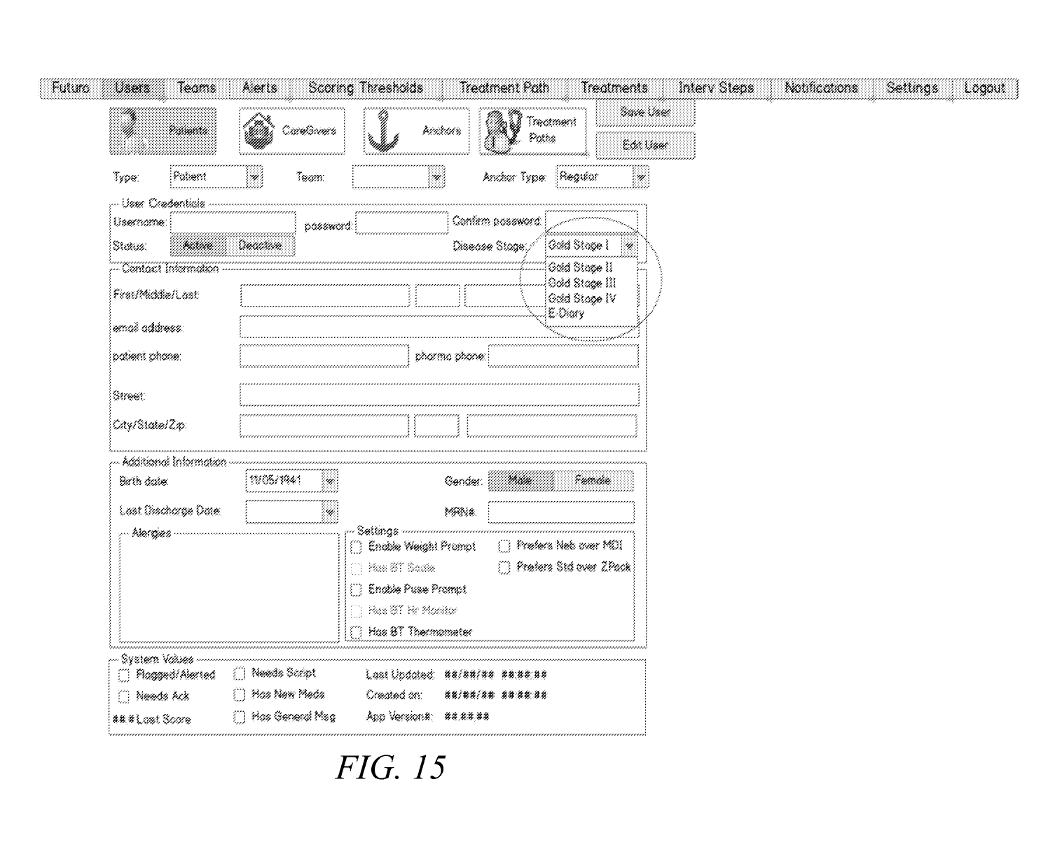

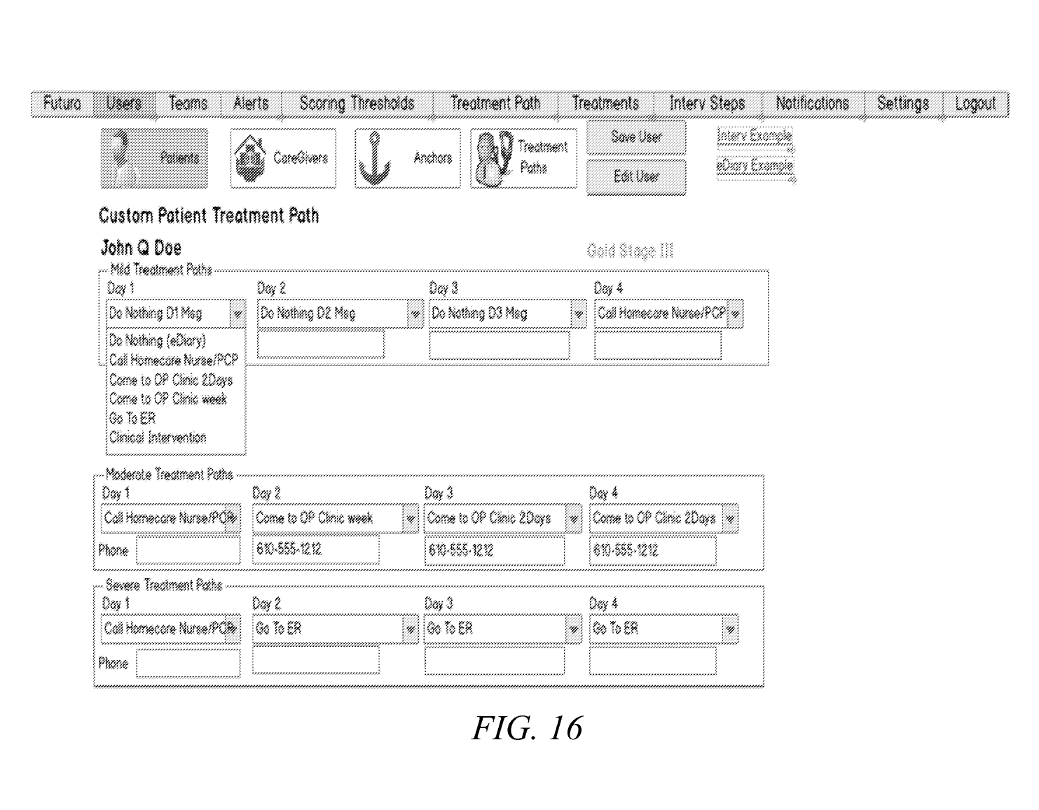

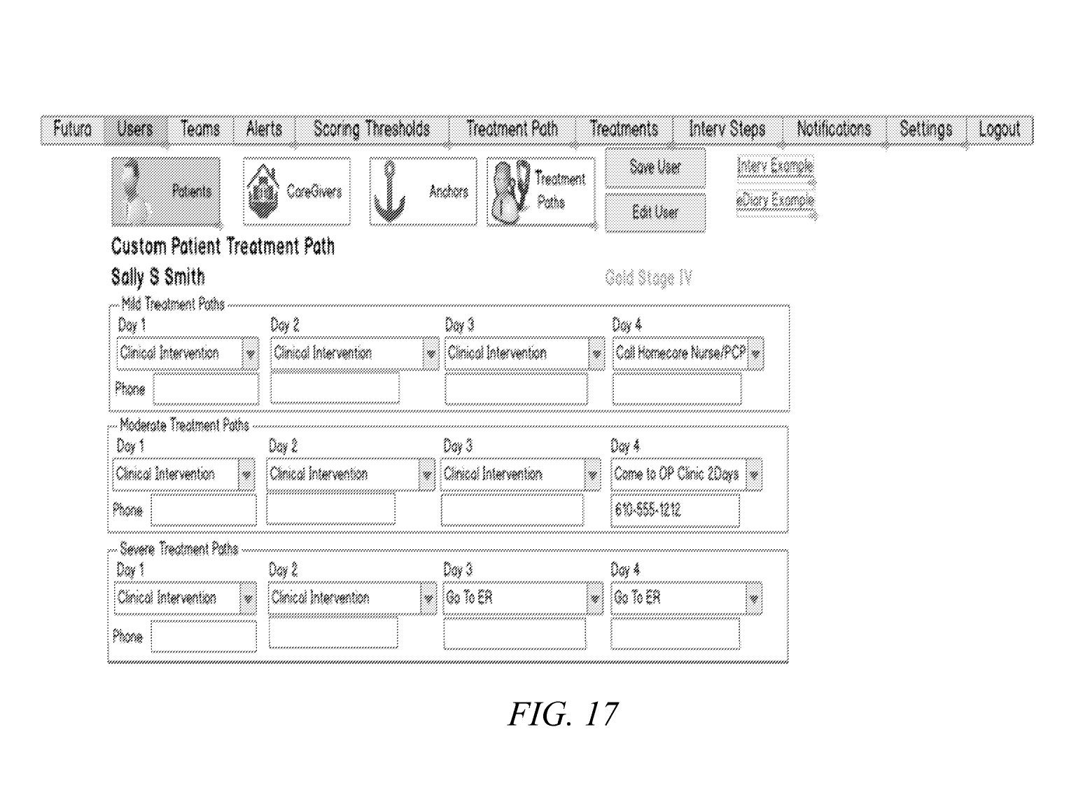

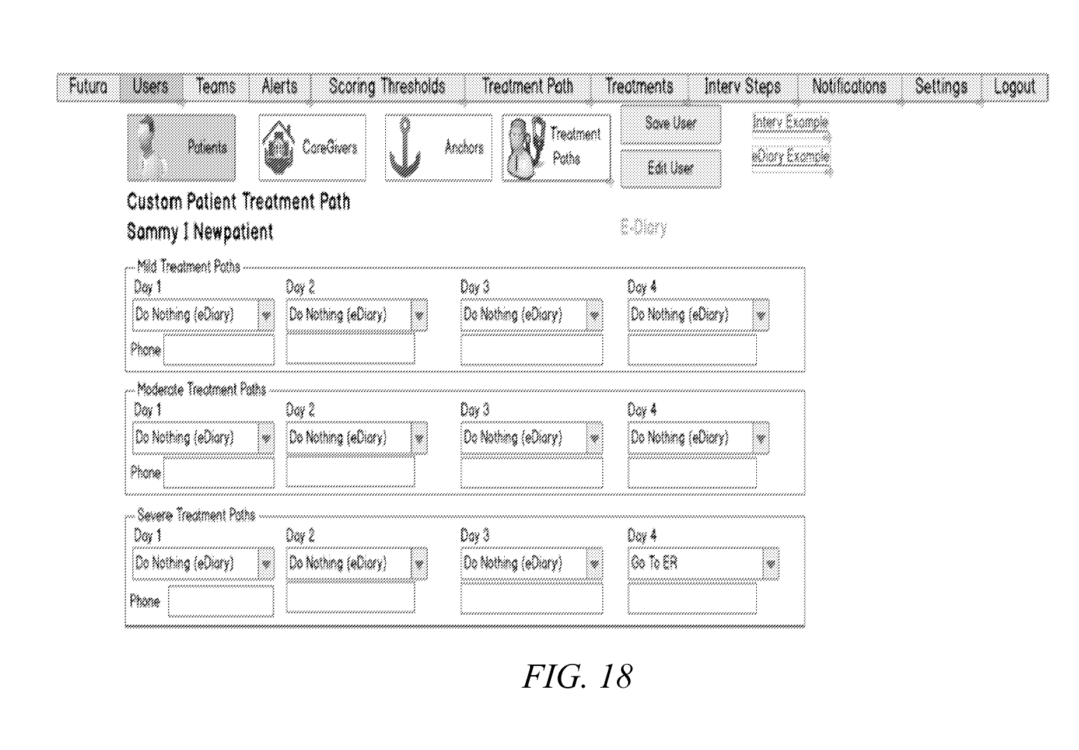

The present system is customizable by the physician, clinician or other authorized personnel on a system-wide basis and on a patient-by-patient basis. For example, FIGS. 7-14 show computer-generated user interfaces for the customization of different parameters involved in facilitating an intervention on behalf of a patient experiencing an exacerbation on a system-wide level. The parameters established on this level are used as the default parameters for each patient onboarded into the system. However, patient-specific parameters customized for individual patients as described below with reference to FIGS. 15-18 supersede the default parameters entered at the system-wide level as described with reference to FIGS. 7-14. FIGS. 7 and 8 show illustrative embodiments of computer-generated displays that facilitate the configuration of notifications to be transmitted by the system 10 in the event of an intervention. As shown in FIG. 7, a plurality of different categories/types of notifications 100 are presented along with the desired recipient(s) 102 who shall receive the respective notifications in the event a corresponding intervention occurs (e.g., before, during, and/or after an intervention).

Creation of the notifications 100 populating the Intervention Notifications List 104 is accomplished by selecting the “Create” button 105 to access the Notification Creation interface 106 shown in FIG. 8. In addition to specifying the desired name 110 and adding one or more desired recipients 112 who are to be notified in the event a corresponding intervention occurs, customized content of the notification can be entered as free text in the text field 114. The desired recipients 112 can be selected from a menu of available entries that have been pre-verified as authorized to receive communications containing potentially-sensitive information about the patient's health (e.g., in compliance with HIPAA and other such regulations governing the privacy and integrity of medical information). The contact information used to transmit the notification text to the selected recipients can be saved in the system.

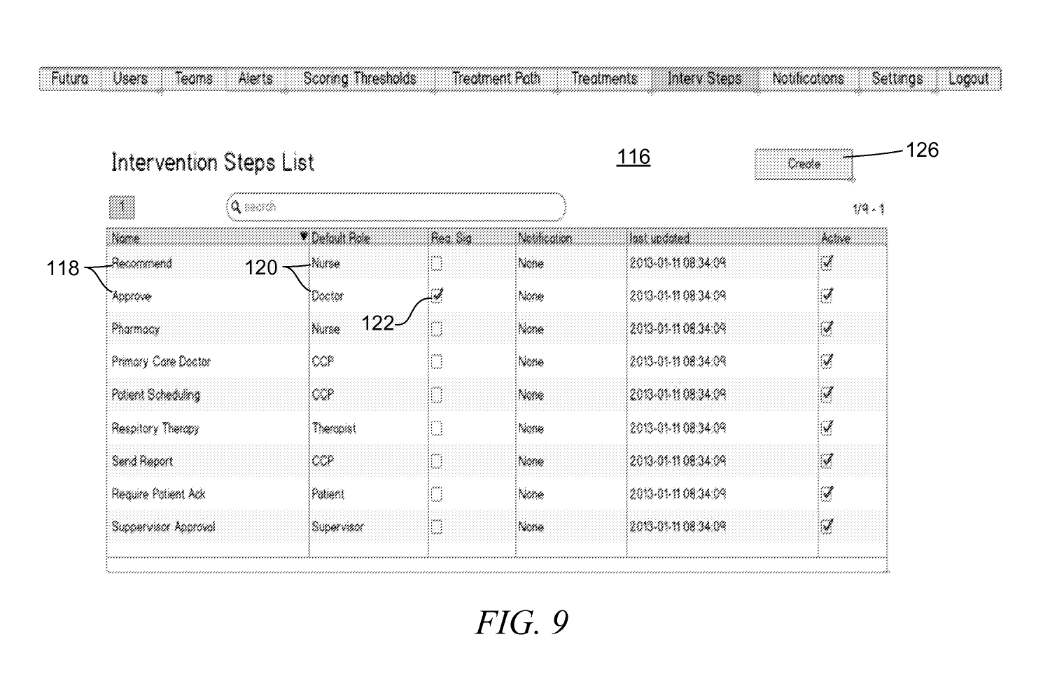

Similarly, in addition to the creation of custom notifications, individual steps to be performed by various different parties in the event of an intervention can also be arranged in the order in which they are to be performed for a given intervention. As shown in the Intervention List 116 of FIG. 9, each step 118 is presented in the order in which it is to be performed, and identifies the person 120 who is to perform the step. As explained above, for a linear workflow each step must be completed before the next step can be performed. A check box 122 can be selected to require the party performing a respective step to sign or otherwise enter confirmation that their step has been completed.

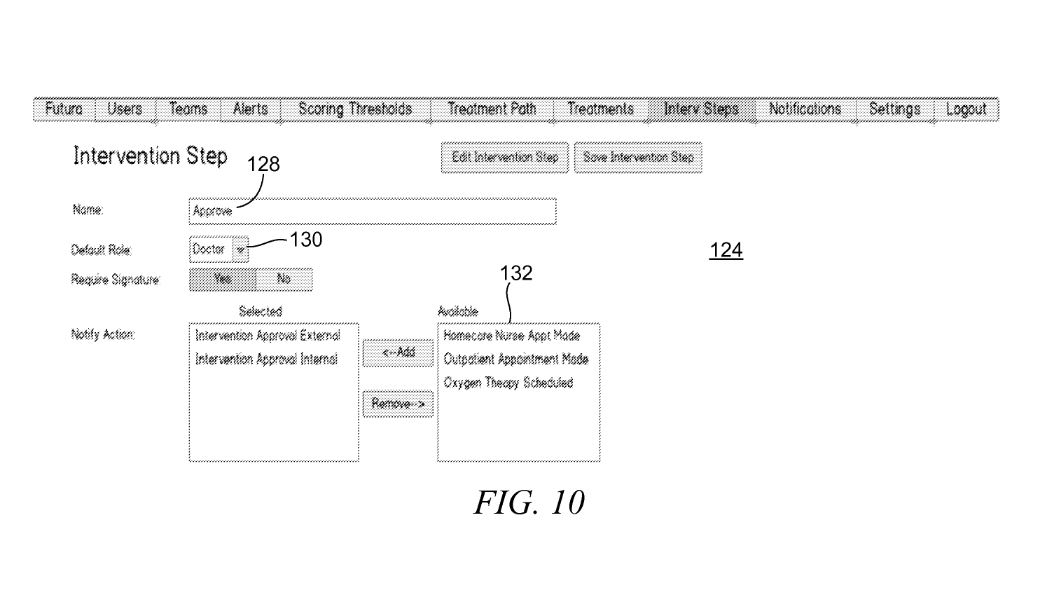

Similar to the notifications, the steps 118 can be created by accessing a Step Creation interface 124 (FIG. 10) by selecting the “Create” button 126. Again, each step is given a name 128, and the party responsible 130 is assigned. Notifications created in the system are selected from a menu 132 and designated to be transmitted in response to, during, or prior to the performance of each step 118.

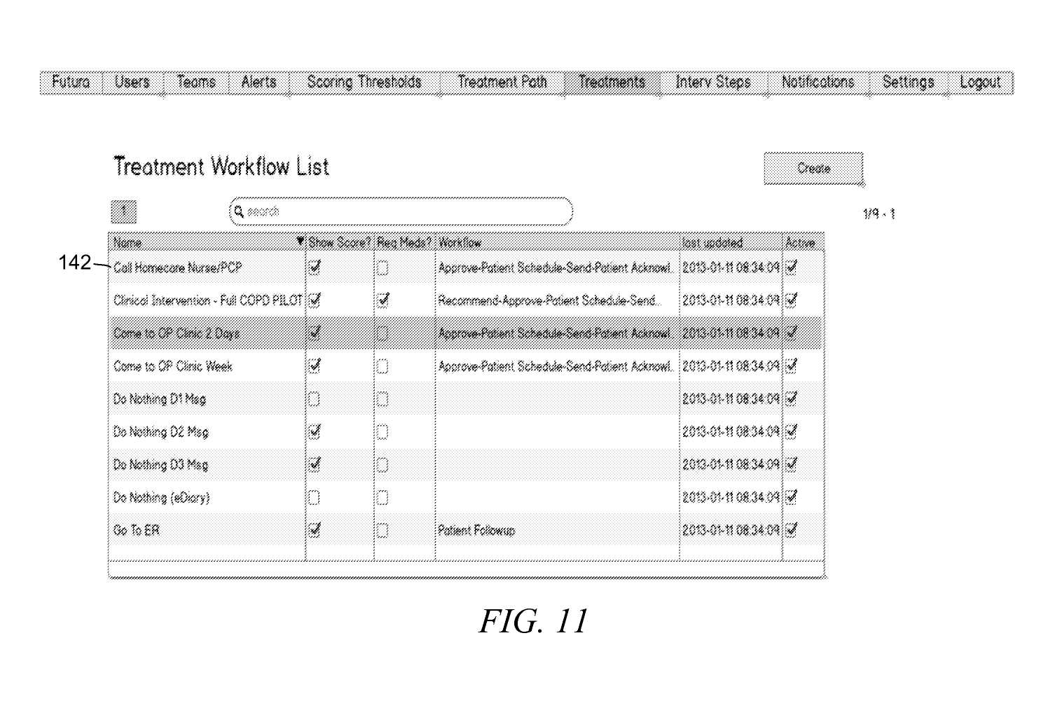

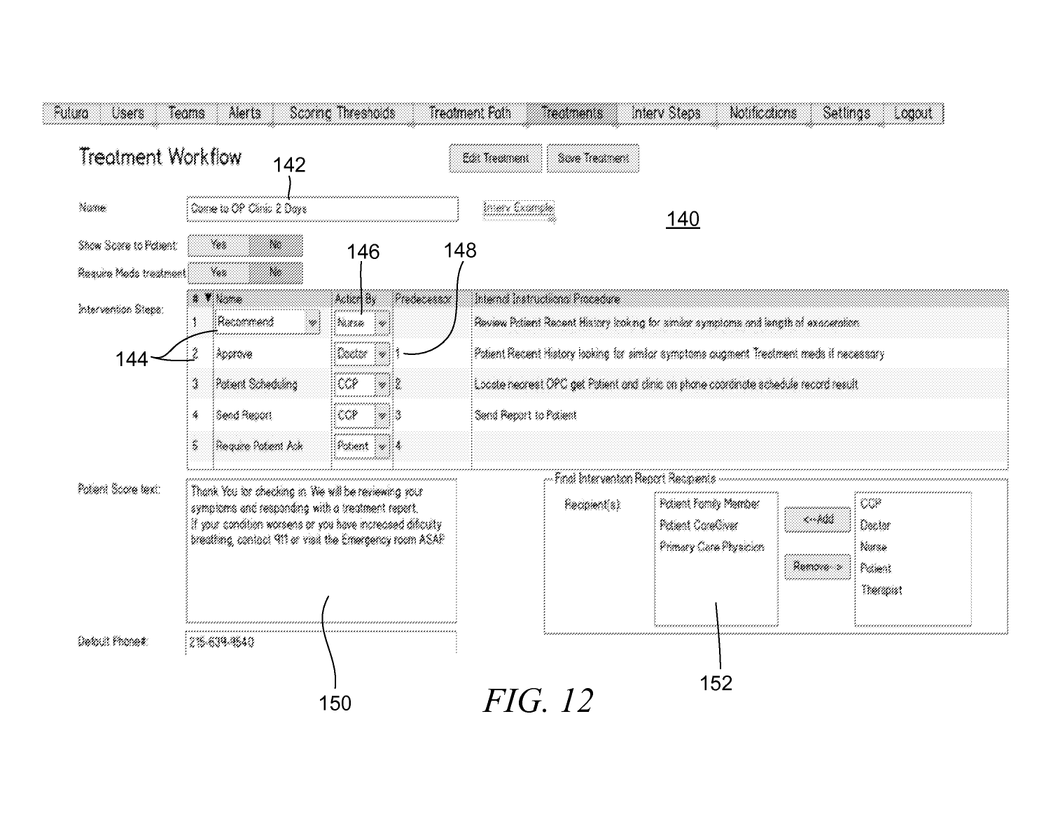

FIG. 11 shows an illustrative embodiment of a workflow list including a plurality of different treatment workflows created via the Treatment Creation interface 140 appearing in FIG. 12. Each treatment workflow is given a name 142, the steps 144 to be performed, the person 146 who is to perform each step 144, along with the predecessor 148 step that is required to be completed immediately before each step 144 can be performed. A custom text reply 150 to be transmitted to the patient in response to receiving information submitted by the patient while checking in and the recipient(s) 152 of the final intervention report summarizing the intervention are also specified in the Treatment Creation interface 140.

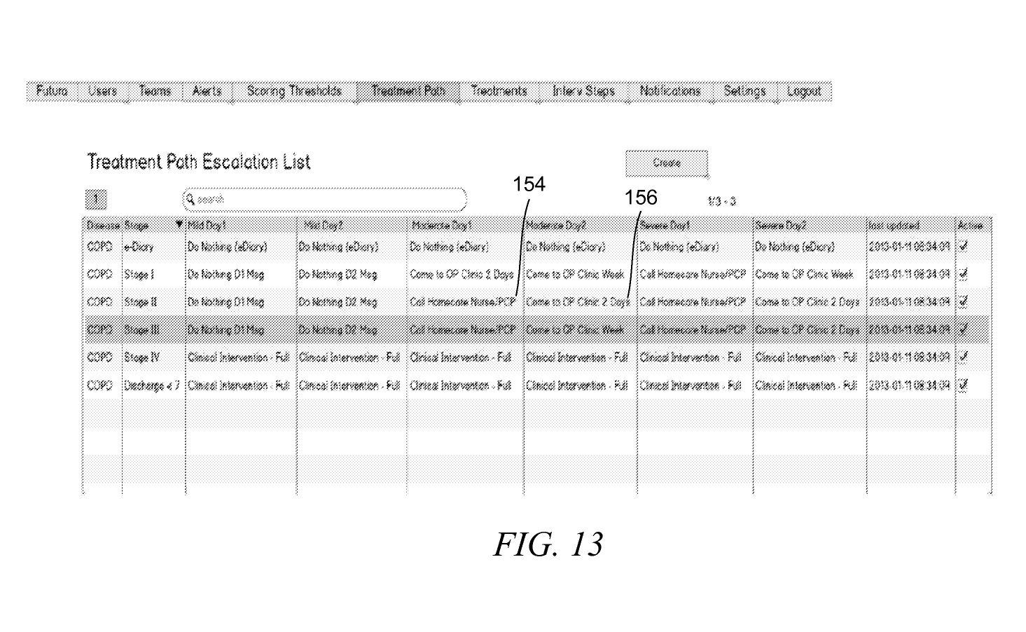

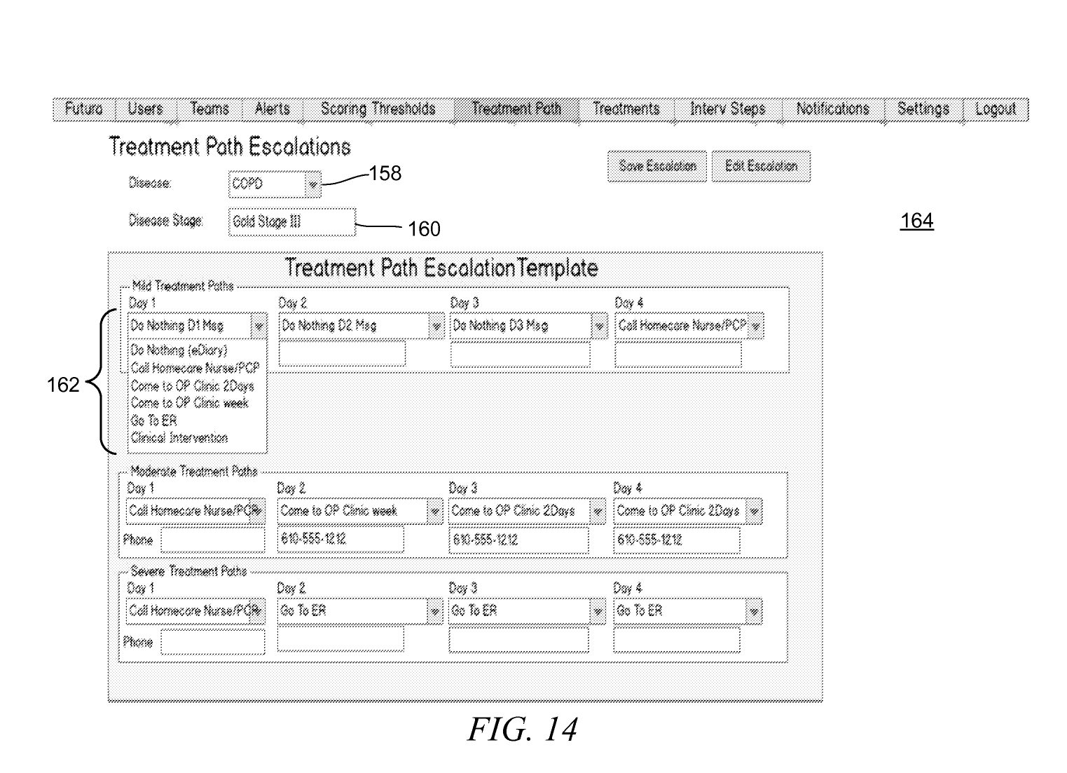

Patients may experience symptoms of an exacerbation that, when considered by itself, would not be cause for concern. However, if such symptoms are not alleviated in a timely manner and persist for extended period of time, the treatment recommended via the present system can be escalated according to an escalation schedule as shown in FIG. 13 and created in FIG. 14. For example, the physician can specify that a patient with Stage II COPD should be presented with the “Call the Homecare Nurse/PCP” treatment path 154 (FIG. 13) on the first day a moderate exacerbation is experienced. If, however, the moderate exacerbation persists into a second day, the “Come to OP Clinic 2 Days” option 156 should be recommended. The disease 158, stage 160, and the recommended options 162 for each time period can be specified in the Escalation interface 164 shown in FIG. 14.

As mentioned above, the parameters established according to the description of FIGS. 7-14 are default values established for a given physician for all patients under that physician's care that are onboarded, but for whom other, custom values of the parameters are not separately established. In contrast, FIGS. 15-18 show computer-generated interfaces that allow separate, patient-specific values of the parameters to be established for individual patients. In other words, the patient-specific values supersede any of the default values established as described with reference to FIGS. 7-14. Any remaining values that are not superseded, however, can be assigned the default values. Thus, special circumstances unique to a given patient can be accommodated.

The following is a non-limiting example of a Physician in a small outpatient clinical setting treating a patient in a disease stage of GOLD Class II. The patient will first be onboarded into the system and will be identified by their disease stage as well as other variables the physician would like to monitor while under their care. The physician will then set the symptom thresholds predictive of an acute exacerbation for the patient as well as the check-in anchor range. The treatment pathway will then be configured for the patient based upon limiting and non-limiting variables. Lastly, the physician will decide upon the information flow, as well as assign roles of observing and acting parties. Once the aforementioned configurations are set, the patient may begin the monitoring process. The patient will check-in daily and if the patient is configured in a stagnant system, the patient will need interventional treatment/therapy only if their scores emit an exacerbation score. If the patient is in a continuous system, the patient will be monitored daily by a clinician and may receive interventional therapy/treatment regardless of their corresponding score.

In a non-limiting example of a Physician in a hospital system setting treating a patient in a disease stage of GOLD Class II, the patient will be onboarded, disease stage identified, symptom thresholds will be set, treatment pathways configured, and information flow decided. A vast difference expected in the configurable system is the information flow, treatment pathways, and configurable roles of all parties involved. Due to a larger patient population present in this outpatient clinical setting scenario, there needs to be definitive patient cohorts, as well as roles assigned to all parties. In a non-limiting example of a patient in a hospital system, the patient may move within the hospital system (e.g., from ER to outpatient clinic) due to many factors (location/staff support, etc. . . . ) so there is a clinical staffperson overseeing them through the transitional period. Certain clinicians have the ability to transition between hospitals/clinics to fill needs/vacancies. This allows for a more responsive and reactive system. In addition, with a hospital system, there may be several outpatient or readycare clinics that the patient may use for emergent reasons. Thus, the treatment pathways may have variables such as location (GPS) to make the information flow reflect available personnel at different geographic locations.

In another non-limiting example, the data needs to be actively and retroactively assessed to analyze the effectiveness of the treatment pathways. Furthermore, this analysis needs to be done at the program level at large as well at the individual patient level. To further the notion of comprehensive coordinated care, patient outcome data, patient risk stratification, and financial assessment of cost to treat COPD patients' needs to be utilized.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A system for assessing a condition of a patient with chronic condition, the system comprising:
a non-transitory computer-readable storage device that stores a plurality of variables relating to symptoms being experienced by the patient during an exacerbation of the chronic condition;
a baseline component that is operable to establish a baseline indicative of a normal condition of the patient with the chronic condition; and
a risk assessment component that is operable to compare the condition of the patient as determined based on the variables to the baseline to determine a severity of the exacerbation of the chronic condition relative to the baseline.

2. The system of claim 1, wherein the baseline established by the baseline component is indicative of a likelihood that another exacerbation will be experienced by the patient in the future.

3. The system of claim 1, wherein the computer-readable device stores a rule that results in transmission of a signal in response to receiving the variables and a determination that an exacerbation has a severity above a threshold severity.

4. The system of claim 3, wherein the threshold severity is customizable by a medical professional, and is based on a history of the patient's health.

5. The system of claim 1, wherein the plurality of variables are entered into a portable computer terminal and received over a communication network from a remote location.

6. The system of claim 1 further comprising a workflow component that transmits an alert to a clinician in response to storage of the plurality of variables in the non-transitory computer storage device and a determination of the severity of the exacerbation.

7. The system of claim 6, wherein the workflow component comprises an approval component that receives, from a medical professional, written approval of a course of treatment recommended by the clinician.

8. The system of claim 7, wherein the workflow component comprises a treatment component that receives an alternative course of treatment mandated by a medical professional instead of a course of treatment recommended by the clinician.

9. The system of claim 1, wherein the baseline is established based at least in part on a result of an in-person examination of the patient by a medical professional.

10. The system of claim 1 further comprising a stagnant workflow component comprising a notification component that automatically, without manual user intervention, transmits a notification over the communication network to be received by the patient, instructing the patient to seek medical attention.
